(19) United States
(12) Patent Application Publication
(10) Pub. No.: US 2023/0089273 A1
(43) Pub. Date: Mar. 23, 2023

(54) METHODS FOR MATURING AN ARTERIOVENOUS FISTULA

(71) Applicant: ALUCENT BIOMEDICAL, INC., Salt Lake City, UT (US)

(72) Inventor: Katalin KAUSER, Salt Lake City, UT (US)

(73) Assignee: ALUCENT BIOMEDICAL, INC., Salt Lake City, UT (US)

(21) Appl. No.: 17/796,245

(22) PCT Filed: Jan. 28, 2021
(86) PCT No.: PCT/US2021/015439

§ 371 Date: Jul. 28, 2022

Related U.S. Application Data
(60) Provisional application No. 62/967,465, filed on Jan. 29, 2020.

(51) Int. Cl. A61M 1/36; A61L 31/16

(52) U.S. Cl. CPC A61M 1/3655; A61L 31/16

(57) ABSTRACT

The present disclosure provides novel methods of maturing an AVF at the time of fistula creation comprising administering to an AVF a composition comprising at least one active agent chosen from a Compound of Formula (I) and pharmaceutically acceptable salts thereof and illuminating the at least one active agent on said AVF with visible light, such as, for example, 450 nm light.

This application claims the benefit of priority of U.S. Provisional Patent Application No. 62/967,465, filed Jan. 29, 2020, which is incorporated herein by reference.

End stage renal disease (ESRD) is characterized by complete or near complete failure of kidney function. For patients with ESRD, hemodialysis is a commonly used therapy in which the patient's blood is removed, filtered through a machine, and returned to the body. Vascular access for hemodialysis may be provided, for example, by an arteriovenous fistula (AVF).

An AVF is created by surgically connecting an artery to a vein. Once created, the high blood pressure of the artery causes increased blood flow to the vein. The forces of pulsatile pressure initiate a remodeling response referred to as maturation. An AVF must mature before it can tolerate routine cannulation for dialysis. Successful maturation requires that the venous vessel walls thicken by outward remodeling, meaning that the lumen size remains substantially unchanged.

The forces of pulsatile pressure associated with AVF creation, however, may damage the extracellular matrix at the luminal wall. This can increase proliferative signals, which can lead to inward stenotic processes that decrease lumen size. AVF maturation failure is caused by luminal stenosis due to excessive neointimal hyperplasia and/or impaired outward remodeling. AVF maturation is unsuccessful not only if the vessel wall does not thicken, but also if vessel wall thickening is accompanied by a decrease in lumen size. If maturation fails, the surgical procedure must be repeated.

Among many different biological processes, medial fibrosis has been previously overlooked, but it is a critical contributor to poor AVF maturation. Excessive fibrosis of the wall due to local inflammation as well as the postoperative angle of fibers have shown positive association with AVF non-maturation. Therapeutic approaches targeting the ECM have been evaluated in the AVF setting previously. For example, topical administration of recombinant human elastase has tested the hypothesis that degradation of elastin in the vessel wall may allow greater early dilation and the elastin fragments may have chemotactic properties redirecting migration of myofibroblasts thereby enhancing outward proliferation. However, testing of this therapeutic principle in phase II and III clinical trials has not shown benefit.

The present disclosure provides novel methods for maturing an AVF comprising treating an arteriovenous fistula with at least one active agent chosen from the Compound of Formula (I):

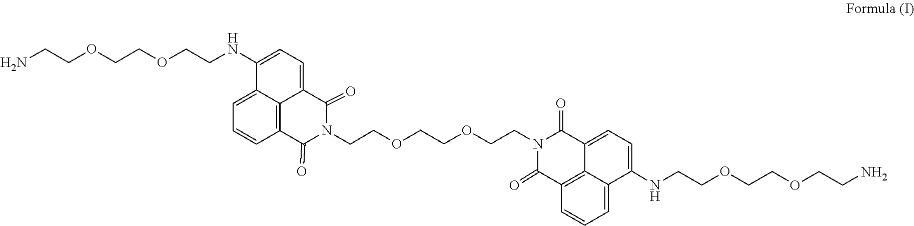 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

and pharmaceutically acceptable salts thereof. In some embodiments, the at least one active agent is administered to the AVF at the time of fistula creation. In some embodiments, the methods comprise administering to the AVF a composition comprising at least one active agent chosen from a Compound of Formula (I) and pharmaceutically acceptable salts thereof and illuminating the at least one active agent on said AVF with visible light, such as, for example, 450 nm light.

It has been surprisingly and unpredictably discovered that treatment of an AVF with at least one active agent chosen from the Compound of Formula (I) and pharmaceutically acceptable salts thereof results in a significant increase in AVF open lumen area, without significantly affecting the area of neointimal hyperplasia. Without being bound by theory, it appears the treatment protects the extracellular matrix at the luminal wall from damage by the increased pulsatile pressure. This decreases proliferative signals at the luminal wall leading to increased outward remodeling.

The present disclosures present a novel technology which helps to preserve the natural ECM scaffold using photochemical activation by a small molecule and 450 nm light during the process of vascular dilation by angioplasty balloon. During photoactivation, the ECM fibers are relinked at a position they have been stretched by the balloon inflation. Durable covalent bonds form between amino acids of collagen and elastin fibers, which help retain the enlarged lumen size, but remain flexible to intravascular pressure changes unlike stent implants. This scaffolding effect helps to reduce the sudden hemodynamic impact by the changing blood flow and pressure the vein receives following fistula creation and indirectly contributes to the regulation of subsequent cellular responses.

### BRIEF DESCRIPTION OF DRAWINGS

This application file contains at least one drawing executed in color. Copies of this patent application publication with color drawing(s) will be provided by the Office upon request and payment of the necessary fee.

FIG. 1A shows an illustration of the surgical procedure described in the Example 1.

FIG. 1B shows a photograph after the surgical procedure described in the Example 1.

FIGS. 1C-E show photographs of the surgical procedure described in Example 1. (C) AVF surgery area was incubated with NVS solution for 5 min; (D) NVS was activated by laser at 450 nm for 1 min; (E) Fluorescent images of native blood vessels that are harvested immediately after the NVS and laser treatment. Green fluorescence indicates NVS penetration throughout the anastomosis area.

FIG. 2 shows VVG images of control rat.

FIG. 3 shows VVG images of rat treated with Compound of Formula (I).

FIGS. 4A-H show representative images of hematoxylin and eosin (H&E) staining (A, C, E, G) and Verhoeff-Van gieson (VVG) staining (B, D, F, H) of the AVF anastomoses at 1 week (A, B, E, F) and 4 weeks (C, D, G, H) from animals treated with phosphate buffered saline (“PBS”) (A-D) and NVS (E-H). Scale bar =100 μm. A=Arterial limb of AVF. V=Venous limb of AVF. L=Lumen. NL=Neointimal lesion.

FIG. 5 shows rat 4-week AVF morphometric analysis data for the total AVF, the IEL-enclosed area, Open lumen area, NH area, and Fraction of open lumen in no-NVS and NVS groups.

FIGS. 6A-D show rat 1-week and 4-week AVF morphometric analysis data for the AVF vein, the IEL-enclosed area, open lumen area, NH area, and percentage of open lumen in no-NVS (PBS) and NVS groups. Results are presented as average±standard deviation. N=3-4 per group in week 1. N=6-9 per group in week 4. *p<0.05 **<0.01.

FIGS. 7A-D shows rat 1-week and 4-week AVF morphometric analysis data for the AVF artery, the IEL-enclosed area, open lumen area, NH area, and percentage of open lumen in no-NVS (PBS) and NVS groups. Results are presented as average±standard deviation. N=3-4 per group in week 1. N=6-9 per group in week 4. *p<0.05 **<0.01.

FIGS. 8A-D show representative immunohistochemistry images of MMP2 staining (A, B) of the AVF anastomoses at 4 weeks from animals treated with PBS (A) and NVS (B); the quantification of staining intensity (i.e., mean gray value) (C); and the quantification of the percentage area stained positive (D). Results are presented as average±standard error of mean. White arrows indicate example regions of positive staining. N=3 per group. *p<0.05. Scale bar=100 A=Arterial limb of AVF. V=Venus limb of AVF. L=Lumen. NL=Neointimal lesion.

FIGS. 9A-D show presentative immunohistochemistry images of MMP-9 staining (A, B) of the AVF anastomoses at 4 weeks from animals treated with PBS (A) and NVS (B); the quantification of staining intensity (i.e., mean gray value) (C); and the quantification of the percentage area stained positive (D). Results are presented as average±standard error of mean. White arrows indicate example regions of positive staining. N=3 per group. *p<0.05. Scale bar=100 μm. A=Arterial limb of AVF. V=Venus limb of AVF. L=Lumen. NL=Neointimal lesion.

FIGS. 10A-D show representative immunohistochemistry images of IL-6 (A, B) staining of the AVF anastomoses at 4 weeks from animals treated with PBS (A) and NVS (B); the quantification of staining intensity (i.e., mean gray value) (C); and the quantification of the percent area stained positive (D). Analyses were performed for the whole AVF, venous limb, and arterial limb. Results are presented as average±standard error of mean. N=3 per group. *p<0.05. Scale bar=100 μm. A=Arterial limb of AVF. V=Venus limb of AVF. L=Lumen. NL=Neointimal lesion. White arrows indicate example regions of positive staining.

FIGS. 11A-D show representative immunohistochemistry images of Ki67 (A, B) staining of the AVF anastomoses at 4 weeks from animals treated with PBS (A) and NVS (B); the quantification of staining intensity (i.e., mean gray value) (C); and the quantification of the percent area stained positive (D). Analyses were performed for the whole AVF, venous limb, and arterial limb. Results are presented as average ±standard error of mean. N=3 per group. Scale bar=100 μm. A=Arterial limb of AVF. V=Venus limb of AVF. L=Lumen. NL=Neointimal lesion. White arrows indicate example regions of positive staining.

FIGS. 12A-B show HUVEC apoptosis (A) and HASMC apoptosis (B) of cultured cells upon exposure to NVS. Results are presented as average±standard deviation.

FIG. 13A-D show an analysis of non-surgery blood vessels. Representative VVG images of contralateral, no-surgery femoral artery and vein from animals treated with PBS (A) and NVS (B). C and D show IEL-enclosed area of the non-surgery vein (C) and non-surgery artery (D), and compare them to the IEL-enclosed area of the AVF venous limbs and arterial limbs, respectively. Results are presented as average±standard error of mean. N=8-9 in each of the non-surgery artery and vein groups. *p<0.001. **p<0.005. A=Artery. V=Vein. L=Lumen.

FIG. 14A-D shows an analysis of collagen fibers in AVF. Representative multiphoton autofluorescence (green) and SHG (red) images of AVFs at 4 weeks from animals treated with PBS (A, B) and NVS (C, D). B and D show the enlarged regions of interested used for the analysis of fiber structure.

As used herein, the following definitions shall apply unless otherwise indicated.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “maturation” of an AVF refers to an increase in AVF open lumen area without significantly affecting the area of neointimal hyperplasia. A “mature” AVF has sufficient open lumen area (i.e. outward remodeling) and limited neointimal hyperplasia (i.e. inward remodeling) to allow increases in AVF blood flow for dialysis. In some embodiments, the increase in a mature AVF open lumen area occurs without significantly decreasing the compliance of the vessel.

As used herein “Natural Vascular Scaffolding” or “NVS” treatment or therapy refers to treatment of a substrate using at least one active agent, such as at least one active agent chosen from the Compound of Formula (I) and pharmaceutically acceptable salts thereof according to the present disclosure. NVS treatment interlinks collagen and elastin by covalently linking these proteins via photoactivation. Similarly, as used herein, a “Natural Vascular Scaffolding” or “NVS” solution refers to a solution comprising at least one active agent, such as at least one active agent chosen from the Compound of Formula (I) and pharmaceutically acceptable salts thereof according to the present disclosure.

As used herein, “phosphate buffered saline” or “PBS” treatment or solution refers to treatment with or solution of phosphate buffered saline that, unless indicated, does not comprise at least one active agent as defined herein. As used herein, a substrate or subject treated with “phosphate buffered saline” or “PBS” would be in a “no-NVS” group.

The “at least one active agent” is chosen from dimeric naphthalimide compounds. Certain dimeric naphthalimide compounds have been previously disclosed. See, e.g., U.S. Pat. No. 6,410,505 B2. For example, a dimeric naphthalimide compound, 2,2′-((ethane-1,2-diylbis(oxy))bis(ethane-2,1-diyl))bis(6-(2-(2-(2-aminoethoxy)ethoxy)ethyl)amino)-1H-benzo[de]isoquinoline-1,3(2H)-dione), also known as 10-8-10 dimer, 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin yl]ethoxy]ethoxy]ethyl]benzo[de]isoquinoline-1,3-dione; 2,2′-[1,2-ethanediylbis(oxy-2,1-ethanediyl)]bis[6-({2-[2-(2-aminoethoxy)ethoxy]ethyl}amino)-1H-benzo[de]isoquinoline-1,3(2H)-dione]; and 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9Cl), and herein referred to as Compound of Formula (I), has been disclosed. Id.

In some embodiments, the at least one active agent is chosen from the Compound for Formula (I) and pharmaceutically acceptable salts thereof. The “Compound of Formula (I)” as used herein may be described by the structure:

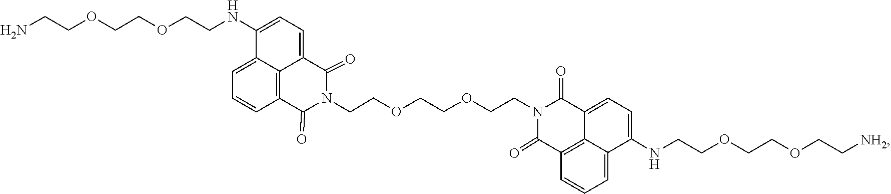 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

by the chemical names 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione); 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbis(oxy-2,1-ethanediyl)]bis[6-({2-[2-(2-aminoethoxy)ethoxy]-ethyl}amino)-1H-benzo[de]isoquinoline-1,3(2H)-dione]; or 1H-benz[de]isoquinoline-1,3(2H)-dione, 2,2′-[1,2-ethanediylbis(oxy-2, 1-ethanediyl)]bis[6-[[2-[2-(2-aminoethoxy)ethoxy]ethyl]amino]-(9Cl), or by the Chemical Abstract Services (CAS) Registry No. 438200-66-9.

As used herein, “Compound of Formula (I)” includes one or more of the conformational forms of the compound. Unless stated otherwise, compounds depicted herein coexisting with tautomeric forms are within the scope of the disclosure. Additionally, unless stated otherwise, structures depicted herein are also meant to include compounds that differ only in the presence of one or more isotopically enriched atoms. For example, compounds having the depicted structures except for the replacement of hydrogen by deuterium or tritium, or the replacement of a carbon atom by ¹³C- or ¹⁴C-enriched carbon atom are within the scope of this disclosure.

### The At Least One Active Agent

In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate. In some embodiments, the at least one active agent is 2,2′4((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

### Compositions

In some embodiments, the methods provided herein comprise administration of a composition comprising at least one active agent to an AVF. In some embodiments, the at least one active agent is present in an amount ranging from 0.01% to 5% by weight of the composition. In some embodiments, the at least one active agent is present in an amount ranging from 0.01% to 4% by weight of the composition. In some embodiments, the at least one active agent is present in an amount ranging from 0.01% to 2.5% by weight of the composition. In some embodiments, the at least one active agent is present in an amount of 0.01%, 0.03%, 0.05%, 0.07%, 0.09%, 0.11%, 0.13%, 0.15%, 0.17%, 0.19%, 0.21%, 0.23%, 0.25%, 0.27%, 0.29%, 0.31%, 0.33%, 0.35%, 0.37%, 0.39%, 0.41%, 0.43%, 0.45%, 0.47%, 0.49%, 0.51%, 0.53%, 0.55%, 0.57%, 0.59%, 0.61%, 0.63%, 0.65%, 0.67%, 0.69%, 0.71%, 0.73%, 0.75%, 0.77%, 0.79%, 0.81%, 0.83%, 0.85%, 0.87%, 0.89%, 0.91%, 0.93%, 0.95%, 0.97%, 0.99%, 1.01%, 1.03%, 1.05%, 1.07%, 1.09%, 1.11%, 1.13%, 1.15%, 1.17%, 1.19%, 1.21%, 1.23%, 1.25%, 1.27%, 1.29%, 1.31%, 1.33%, 1.35%, 1.37%, 1.39%, 1.41%, 1.43%, 1.45%, 1.47%, 1.49%, 1.51%, 1.53%, 1.55%, 1.57%, 1.59%, 1.61%, 1.63%, 1.65%, 1.67%, 1.69%, 1.71%, 1.73%, 1.75%, 1.77%, 1.79%, 1.81%, 1.83%, 1.85%, 1.87%, 1.89%, 1.91%, 1.93%, 1.95%, 1.97%, 1.99%, 2.01%, 2.03%, 2.05%, 2.07%, 2.09%, 2.11%, 2.13%, 2.15%, 2.17%, 2.19%, 2.21%, 2.23%, 2.25%, 2.27%, 2.29%, 2.31%, 2.33%, 2.35%, 2.37%, 2.39%, 2.41%, 2.43%, 2.45%, 2.47%, or 2.49% by weight of the composition. In some embodiments, the at least one active agent is present in an amount of 0.1%, 0.2%, 0.3%, 0.4%, 0.5%, 0.6%, 0.7%, 0.8%, 0.9%, 1%, 1.1%, or 1.2% by weight of the composition.

In some embodiments, the at least one active agent is present in an amount ranging from 0.01% to 1% by weight of the composition. In some embodiments, the at least one active agent is present in an amount ranging from 0.3% to 0.6% by weight of the composition. In some embodiments, the at least one active agent is present in an amount ranging from 0.4% to 0.5% by weight of the composition. In some embodiments, the at least one active agent is present in an amount ranging from 0.1% to 0.5% by weight of the composition. In some embodiments, the at least one active agent is present in an amount of 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, or 0.5% by weight of the composition. In some embodiments, the at least one active agent is present in an amount ranging from 0.1% to 0.3% by weight of the composition.

In some embodiments, the at least one active agent is present in an amount of 0.01%, 0.02%, 0.03%, 0.04%, 0.05%, 0.06%, 0.07%, 0.08%, 0.09%, 0.1%, 0.12%, 0.14%, 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.48%, 0.5%, 0.52%, 0.54%, 0.56%, 0.58%, 0.6%, 0.62%, 0.64%, 0.66%, 0.68%, 0.7%, 0.72%, 0.74%, 0.76%, 0.78%, 0.8%, 0.82%, 0.84%, 0.86%, 0.88%, 0.9%, 0.92%, 0.94%, 0.96%, 0.98%, or 1% by weight of the composition.

In some embodiments, the at least one active agent is present in an amount of 0.08% by weight of the composition. In some embodiments, the at least one active agent is present in an amount of 0.1% by weight of the composition. In some embodiments, the at least one active agent is present in an amount of 0.12% by weight of the composition. In some embodiments, the at least one active agent is present in an amount of 0.14% by weight of the composition. In some embodiments, the at least one active agent is present in an amount of 0.16% by weight of the composition. In some embodiments, the at least one active agent is present in an amount of 0.18% by weight of the composition. In some embodiments, the at least one active agent is present in an amount of 0.2% by weight of the composition. In some embodiments, the at least one active agent is present in an amount of 0.22% by weight of the composition. In some embodiments, the at least one active agent is present in an amount of 0.24% by weight of the composition. In some embodiments, the at least one active agent is present in an amount of 0.26% by weight of the composition. In some embodiments, the at least one active agent is present in an amount of 0.36% by weight of the composition. In some embodiments, the at least one active agent is present in an amount of 0.38% by weight of the composition. In some embodiments, the at least one active agent is present in an amount of 0.4% by weight of the composition. In some embodiments, the at least one active agent is present in an amount of 0.42% by weight of the composition. In some embodiments, the at least one active agent is present in an amount of 0.44% by weight of the composition. In some embodiments, the at least one active agent is present in an amount of 0.46% by weight of the composition. In some embodiments, the at least one active agent is present in an amount of 0.48% by weight of the composition. In some embodiments, the at least one active agent is present in an amount of 0.5% by weight of the composition.

### At Least One Solvent

In some embodiments, the composition further comprises at least one solvent. In some embodiments, the at least one solvent is chosen from water, ethanol, isopropanol, polyethylene glycols, and propylene glycols.

In some embodiments, the at least one solvent is water. In some embodiments, the at least one solvent is ethanol. In some embodiments, the at least one solvent is isopropanol. In some embodiments, the at least one solvent is chosen from polyethylene glycols. In some embodiments, the at least one solvent is chosen from propylene glycols. In some embodiments, the polyethylene glycols are chosen from polyethylene glycol 300, polyethylene glycol 400, polyethylene glycol 500, and polyethylene glycol 600.

In some embodiments, the at least one solvent is present in an amount of qs 100% by weight of the composition.

### At Least One Tonicity Agent

In some embodiments, the composition further comprises at least one tonicity agent. Without being bound by any theory, in some embodiments, at least one tonicity agent may be added to modulate the solute concentration of a composition.

In some embodiments, the at least one tonicity agent is chosen from dextrose, sorbitol, lactose, mannitol, sodium chloride, potassium chloride, and glycerol. In some embodiments, the at least one tonicity agent is chosen from sodium chloride and potassium chloride. In some embodiments, the at least one tonicity agent is sodium chloride and potassium chloride.

In some embodiments, the at least one tonicity agent is dextrose. In some embodiments, the at least one tonicity agent is sorbitol. In some embodiments, the at least one tonicity agent is lactose. In some embodiments, the at least one tonicity agent is mannitol. In some embodiments, the at least one tonicity agent is sodium chloride. In some embodiments, the at least one tonicity agent is potassium chloride. In some embodiments, the at least one tonicity agent is glycerol.

In some embodiments, the at least one tonicity agent is present in an amount up to 5% by weight of the composition. In some embodiments, the at least one tonicity agent is present in an amount up to 4% by weight of the composition. In some embodiments, the at least one tonicity agent is present in an amount up to 3% by weight of the composition. In some embodiments, the at least one tonicity agent is present in an amount of up to 2.5% by weight of the composition. In some embodiments, the at least one tonicity agent is present in an amount of up to 2% by weight of the composition. In some embodiments, the at least one tonicity agent is present in an amount of up to 1.5% by weight of the composition. In some embodiments, the at least one tonicity agent is present in an amount of up to 1% by weight of the composition. In some embodiments, the at least one tonicity agent is present in an amount of up to 0.5% by weight of the composition.

In some embodiments, the at least one tonicity agent is present in an amount ranging from 0.25% to 3% by weight of the composition. In some embodiments, the at least one tonicity agent is present in an amount ranging from 0.25% to 2.5% by weight of the composition. In some embodiments, the at least one tonicity agent is present in an amount ranging from 0.5% to 2% by weight of the composition. In some embodiments, the at least one tonicity agent is present in an amount ranging from 0.5% to 1.5% by weight of the composition.

In some embodiments, the at least one tonicity agent is present in an amount of 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, or 0.9% by weight of the composition. In some embodiments, the at least one tonicity agent is present in an amount of 0.72%, 0.74%, 0.76%, 0.78%, 0.8%, 0.82%, or 0.84% by weight of the composition. In some embodiments, the at least one tonicity agent is present in an amount of 0.72% by weight of the composition. In some embodiments, the at least one tonicity agent is present in an amount of 0.74% by weight of the composition. In some embodiments, the at least one tonicity agent is present in an amount of 0.76% by weight of the composition. In some embodiments, the at least one tonicity agent is present in an amount of 0.78% by weight of the composition. In some embodiments, the at least one tonicity agent is present in an amount of 0.8% by weight of the composition. In some embodiments, the at least one tonicity agent is present in an amount of 0.82% by weight of the composition. In some embodiments, the at least one tonicity agent is present in an amount of 0.84% by weight of the composition.

### At Least One Buffer

In some embodiments, the composition further comprises at least one buffer. Without being bound by any theory, in some embodiments, at least one buffer may be added to maintain a desired pH or pH range.

In some embodiments, the at least one buffer is chosen from a potassium salt, a sodium salt, and maleic acid. In some embodiments, the at least one buffer is a sodium salt. In some embodiments, the at least one buffer is a potassium salt. In some embodiments, the at least one buffer is maleic acid. In some embodiments, the at least one buffer comprises a potassium salt and a sodium salt.

In some embodiments, the potassium salt is chosen from potassium phosphate, potassium citrate, potassium acetate, potassium lactate, and potassium tartrate. In some embodiments, the sodium salt is chosen from sodium phosphate, sodium citrate, sodium acetate, sodium lactate, and sodium tartrate. In some embodiments, the at least one buffer is chosen from potassium phosphate and sodium phosphate. In some embodiments, the at least one buffer comprises potassium phosphate and sodium phosphate.

In some embodiments, the potassium phosphate is chosen from potassium phosphate monobasic, potassium phosphate dibasic, and potassium phosphate tribasic. In some embodiments, the potassium phosphate is chosen from potassium phosphate monobasic and potassium phosphate dibasic. In some embodiments, the potassium phosphate is potassium phosphate monobasic. In some embodiments, the potassium phosphate is potassium phosphate dibasic. In some embodiments, the potassium phosphate is potassium phosphate tribasic.

In some embodiments, the sodium phosphate is chosen from sodium phosphate monobasic, sodium phosphate dibasic, and sodium phosphate tribasic. In some embodiments, the sodium phosphate is chosen from sodium phosphate monobasic and sodium phosphate dibasic. In some embodiments, the sodium phosphate is sodium phosphate monobasic. In some embodiments, the sodium phosphate is sodium phosphate dibasic. In some embodiments, the sodium phosphate is sodium phosphate tribasic.

In some embodiments, the at least one buffer is chosen from sodium phosphate dibasic and potassium phosphate monobasic. In some embodiments, the at least one buffer is sodium phosphate dibasic and potassium phosphate monobasic.

In some embodiments, the at least one buffer is anhydrous.

In some embodiments, the at least one buffer is present in an amount of up to 2.5% by weight of the composition. In some embodiments, the at least one buffer is present in an amount of up to 2% by weight of the composition. In some embodiments, the at least one buffer is present in an amount of up to 1.5% by weight of the composition. In some embodiments, the at least one buffer is present in an amount of up to 1% by weight of the composition. In some embodiments, the at least one buffer is present in an amount of up to 0.5% by weight of the composition.

In some embodiments, the at least one buffer is present in an amount ranging from 0.05% to 0.4% by weight of the composition. In some embodiments, the at least one buffer is present in an amount of 0.06%, 0.08%, 0.1%, 0.12%, 0.14%, 0.16%, 0.18% 0.2%, 0.22%, 0.24%, 0.26%, 0.28%, or 0.3% by weight of the composition. In some embodiments, the at least one buffer is present in an amount ranging from 0.1% to 0.2% by weight of the composition. In some embodiments, the at least one buffer is present in an amount ranging from 0.08% to 0.16% by weight of the composition. In some embodiments, the at least one buffer is present in an amount of 0.1% by weight of the composition. In some embodiments, the at least one buffer is present in an amount of 0.11% by weight of the composition. In some embodiments, the at least one buffer is present in an amount of 0.12% by weight of the composition. In some embodiments, the at least one buffer is present in an amount of 0.13% by weight of the composition. In some embodiments, the at least one buffer is present in an amount of 0.14% by weight of the composition. In some embodiments, the at least one buffer is present in an amount of 0.15% by weight of the composition. In some embodiments, the at least one buffer is present in an amount of 0.16% by weight of the composition. In some embodiments, the at least one buffer is present in an amount of 0.17% by weight of the composition. In some embodiments, the at least one buffer is present in an amount of 0.18% by weight of the composition. In some embodiments, the at least one buffer is present in an amount of 0.19% by weight of the composition. In some embodiments, the at least one buffer is present in an amount of 0.2% by weight of the composition.

In some embodiments, the at least one buffer comprises sodium phosphate in an amount ranging from 0.01% to 0.03% by weight of the composition and potassium phosphate in an amount ranging from 0.1% to 0.14% by weight of the composition.

### At Least One pH Modulation Agent

In some embodiments, the composition further comprises at least one pH modulation agent. Without being bound by any theory, in some embodiments, at least one pH modulation agent may be added to achieve a desired pH of the composition.

In some embodiments, the at least one pH modulation agent is chosen from acetic acid, carbonic acid, citric acid, sodium bicarbonate, and sodium hydroxide. In some embodiments, the at least one pH modulation agent is acetic acid. In some embodiments, the at least one pH modulation agent is carbonic acid. In some embodiments, the at least one pH modulation agent is citric acid. In some embodiments, the at least one pH modulation agent is sodium bicarbonate. In some embodiments, the at least one pH modulation agent is sodium hydroxide.

In some embodiments, the at least one pH modulation agent is chosen from acetic acid and sodium hydroxide. In some embodiments, the at least one pH modulation agent is acetic acid and sodium hydroxide.

In some embodiments, the at least one pH modulation agent is present in an amount of qs to desired pH.

### At Least One Viscosity Agent

In some embodiments, the composition further comprises at least one viscosity agent. Without being bound by any theory, in some embodiments, at least one viscosity agent may be added to achieve a desired viscosity or thickness of the composition.

In some embodiments, the at least one viscosity agent is chosen from gelatin, methylcellulose, hydroxypropyl cellulose, hydroxypropyl methylcellulose, hydroxyethyl cellulose, hydroxypropylmethyl cellulose, methylhydroxyethyl cellulose, methylhydroxypropyl cellulose, hydroxyethylcarboxymethyl cellulose, carboxymethyl cellulose, carboxymethylhydroxyethyl cellulose, and sodium carboxymethylcellulose. In some embodiments, the at least one viscosity agent is chosen from methylcellulose, sodium carboxymethylcellulose, and hydroxypropyl cellulose.

In some embodiments, the at least one viscosity agent is gelatin. In some embodiments, the at least one viscosity agent is methylcellulose. In some embodiments, the at least one viscosity agent is hydroxypropyl cellulose. In some embodiments, the at least one viscosity agent is hydroxypropyl methylcellulose. In some embodiments, the at least one viscosity agent is hydroxyethyl cellulose. In some embodiments, the at least one viscosity agent is hydroxypropylmethyl cellulose. In some embodiments, the at least one viscosity agent is methylhydroxyethyl cellulose. In some embodiments, the at least one viscosity agent is methylhydroxypropyl cellulose. In some embodiments, the at least one viscosity agent is hydroxyethylcarboxymethyl cellulose. In some embodiments, the at least one viscosity agent is carboxymethyl cellulose. In some embodiments, the at least one viscosity agent is carboxymethylhydroxyethyl cellulose. In some embodiments, the at least one viscosity agent is sodium carboxymethylcellulose.

In some embodiments, the at least one viscosity agent is present in an amount of 2.5% or less by weight of the composition. In some embodiments, the at least one viscosity agent is present in an amount of 2% or less by weight of the composition. In some embodiments, the at least one viscosity agent is present in an amount of 1.5% or less by weight of the composition. In some embodiments, the at least one viscosity agent is present in an amount of 1% or less by weight of the composition. In some embodiments, the at least one viscosity agent is present in an amount of 0.5% or less by weight of the composition.

In some embodiments, the at least one viscosity agent is present in an amount ranging from 0.05% to 1% by weight of the composition. In some embodiments, the at least one viscosity agent is present in an amount ranging from 0.1% to 0.5% by weight of the composition. In some embodiments, the at least one viscosity agent is present in an amount ranging from 0.1% to 0.3% by weight of the composition. In some embodiments, the at least one viscosity agent is present in an amount of 0.15%, 0.16%, 0.17%, 0.18%, 0.19%, 0.2%, 0.21%, 0.22%, 0.23%, 0.24%, or 0.25% by weight of the composition.

In some embodiments, the at least one viscosity agent is present in an amount of 0.15% by weight of the composition. In some embodiments, the at least one viscosity agent is present in an amount of 0.16% by weight of the composition. In some embodiments, the at least one viscosity agent is present in an amount of 0.17% by weight of the composition. In some embodiments, the at least one viscosity agent is present in an amount of 0.18% by weight of the composition. In some embodiments, the at least one viscosity agent is present in an amount of 0.19% by weight of the composition. In some embodiments, the at least one viscosity agent is present in an amount of 0.2% by weight of the composition. In some embodiments, the at least one viscosity agent is present in an amount of 0.21% by weight of the composition. In some embodiments, the at least one viscosity agent is present in an amount of 0.22% by weight of the composition. In some embodiments, the at least one viscosity agent is present in an amount of 0.23% by weight of the composition. In some embodiments, the at least one viscosity agent is present in an amount of 0.24% by weight of the composition. In some embodiments, the at least one viscosity agent is present in an amount of 0.25% by weight of the composition.

### At Least One Penetration Enhancer

In some embodiments, the composition further comprises at least one penetration enhancer. Without being bound by any theory, in some embodiments, at least one penetration enhancer may be added to increase the amount of the at least one active agent delivered to the desired location.

In some embodiments, the at least one penetration enhancer is chosen from benzyl alcohol, diethylene glycol monoethyl ether, caprylic acid, and sodium oleate. In some embodiments, the at least one penetration enhancer is benzyl alcohol. In some embodiments, the at least one penetration enhancer is diethylene glycol monoethyl ether. In some embodiments, the at least one penetration enhancer is caprylic acid. In some embodiments, the at least one penetration enhancer is sodium oleate.

In some embodiments, the at least one penetration enhancer is present in an amount ranging from 0.01% to 1% by weight of the composition. In some embodiments, the at least one penetration enhancer is present in an amount ranging from 0.01% to 0.5% by weight of the composition. In some embodiments, the at least one penetration enhancer is present in an amount ranging from 0.05% to 0.25% by weight of the composition. In some embodiments, the at least one penetration enhancer is present in an amount of 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1% by weight of the composition.

### At Least One Stabilizing Agent

In some embodiments, the composition further comprises at least one stabilizing agent. Without being bound by any theory, in some embodiments, at least one stabilizing agent may be added to retard or completely prevent degradation of the at least one active agent and/or the stabilizing agent may retard or completely prevent the appearance of impurities in the composition.

In some embodiments, the at least one stabilizing agent is chosen from ascorbic acid, butylated hydroxytoluene, citric acid, benzoic acid, and sodium metabisulfite. In some embodiments, the at least one stabilizing agent is ascorbic acid. In some embodiments, the at least one stabilizing agent is butylated hydroxytoluene. In some embodiments, the at least one stabilizing agent is citric acid. In some embodiments, the at least one stabilizing agent is benzoic acid. In some embodiments, the at least one stabilizing agent is sodium metabisulfite.

In some embodiments, the at least one stabilizing agent is present in an amount ranging from 0.005% to 1% by weight of the composition. In some embodiments, the at least one stabilizing agent is present in an amount ranging from 0.005% to 0.25%, 0.5%, 0.75%, or 1% by weight of the composition.

In some embodiments, the at least one stabilizing agent is present in an amount ranging from 0.01% to 1% by weight of the composition. In some embodiments, the at least one stabilizing agent is present in an amount ranging from 0.01% to 0.25%, 0.5%, 0.75%, or 1% by weight of the composition

In some embodiments, the at least one stabilizing agent is present in an amount ranging from 0.1% to 1% by weight of the composition. In some embodiments, the at least one stabilizing agent is present in an amount ranging from 0.1% to 0.25%, 0.5%, 0.75%, or 1% by weight of the composition.

In some embodiments, the at least one stabilizing agent is present in an amount of 0.005%, 0.01%, 0.025%, 0.05%, 0.1%, 0.15%, 0.2%, 0.25%, 0.3%, 0.4%, 0.5%, 0.75%, or 1% by weight of the composition. In some embodiments, the at least one stabilizing agent is present in an amount of 0.005% by weight of the composition. In some embodiments, the at least one stabilizing agent is present in an amount of 0.01% by weight of the composition. In some embodiments, the at least one stabilizing agent is present in an amount of 0.025% by weight of the composition. In some embodiments, the at least one stabilizing agent is present in an amount of 0.05% by weight of the composition. In some embodiments, the at least one stabilizing agent is present in an amount of 0.1% by weight of the composition. In some embodiments, the at least one stabilizing agent is present in an amount of 0.15% by weight of the composition. In some embodiments, the at least one stabilizing agent is present in an amount of 0.2% by weight of the composition. In some embodiments, the at least one stabilizing agent is present in an amount of 0.25% by weight of the composition. In some embodiments, the at least one stabilizing agent is present in an amount of 0.3% by weight of the composition. In some embodiments, the at least one stabilizing agent is present in an amount of 0.4% by weight of the composition. In some embodiments, the at least one stabilizing agent is present in an amount of 0.5% by weight of the composition. In some embodiments, the at least one stabilizing agent is present in an amount of 0.75% by weight of the composition. In some embodiments, the at least one stabilizing agent is present in an amount of 1% by weight of the composition.

### At Least One Solubilizing Agent

In some embodiments, the composition further comprises at least one solubilizing agent. Without being bound by any theory, in some embodiments, at least one solubilizing agent may be added to increase the solubility of the at least one active agent in a vehicle, e.g., water, by forming, e.g., an emulsion.

In some embodiments, the at least one solubilizing agent is chosen from tocopherols, fixed oils, soybean oil, PEG-15 hydroxystearates, polysorbate 20, polysorbate 80, 2-hydroxypropyl-β-cyclodextrin, and γ-cyclodextrin. In some embodiments, the solubilizing agent is chosen from tocopherols. In some embodiments, the solubilizing agent is chosen from fixed oils. In some embodiments, the solubilizing agent is chosen from PEG-15 hydroxystearates. In some embodiments, the solubilizing agent is polysorbate 20. In some embodiments, the solubilizing agent is polysorbate 80. In some embodiments, the solubilizing agent is 2-hydroxypropyl-β-cyclodextrin. In some embodiments, the solubilizing agent is γ-cyclodextrin.

In some embodiments, fixed oils are chosen from corn oil, cottonseed oil, peanut oil, and sesame oil.

In some embodiments, the at least one solubilizing agent is present in an amount ranging from 0.005% to 10% by weight of the composition. In some embodiments, the at least one solubilizing agent is present in an amount ranging from 0.005% to 0.1%, 0.25%, 0.5%, 1%, 1.5%, 2.5%, 5%, 7.5%, or 10% by weight of the composition. In some embodiments, the at least one solubilizing agent is present in an amount ranging from 0.1% to 10% by weight of the composition. In some embodiments, the at least one solubilizing agent is present in an amount ranging from 0.1% to 0.25%, 0.5%, 1%, 1.5%, 2.5%, 5%, 7.5%, or 10% by weight of the composition.

In some embodiments, the at least one solubilizing agent is present in an amount ranging from 1% to 10% by weight of the composition. In some embodiments, the at least one solubilizing agent is present in an amount ranging from 1% to 1.5%, 2.5%, 5%, 7.5%, or 10% by weight of the composition.

In some embodiments, the at least one solubilizing agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one solubilizing agent is present in an amount of 0.005% by weight of the composition. In some embodiments, the at least one solubilizing agent is present in an amount of 0.01% by weight of the composition. In some embodiments, the at least one solubilizing agent is present in an amount of 0.025% by weight of the composition. In some embodiments, the at least one solubilizing agent is present in an amount of 0.05% by weight of the composition. In some embodiments, the at least one solubilizing agent is present in an amount of 0.1% by weight of the composition. In some embodiments, the at least one solubilizing agent is present in an amount of 0.15% by weight of the composition. In some embodiments, the at least one solubilizing agent is present in an amount of 0.2% by weight of the composition. In some embodiments, the at least one solubilizing agent is present in an amount of 0.25% by weight of the composition. In some embodiments, the at least one solubilizing agent is present in an amount of 0.3% by weight of the composition. In some embodiments, the at least one solubilizing agent is present in an amount of 0.4% by weight of the composition. In some embodiments, the at least one solubilizing agent is present in an amount of 0.5% by weight of the composition. In some embodiments, the at least one solubilizing agent is present in an amount of 0.75% by weight of the composition. In some embodiments, the at least one solubilizing agent is present in an amount of 1% by weight of the composition. In some embodiments, the at least one solubilizing agent is present in an amount of 1.5% by weight of the composition. In some embodiments, the at least one solubilizing agent is present in an amount of 2% by weight of the composition. In some embodiments, the at least one solubilizing agent is present in an amount of 2.5% by weight of the composition. In some embodiments, the at least one solubilizing agent is present in an amount of 5% by weight of the composition. In some embodiments, the at least one solubilizing agent is present in an amount of 7.5% by weight of the composition. In some embodiments, the at least one solubilizing agent is present in an amount of 10% by weight of the composition.

### At Least One Encapsulation Agent

In some embodiments, the composition further comprises at least one encapsulation agent. Without being bound by any theory, in some embodiments, at least one encapsulation agent may be added to improve delivery, stability, and/or solubility of the at least one active agent by, e.g., encapsulating the at least active agent in a liposome or lipid particle.

In some embodiments, the at least one encapsulation agent is chosen from 1,2-dimyristoyl-sn-glycero-phosphocholine, 1,2-distearoyl-sn-glycero-3-(phosphor-rac-(1-glycerol)), 1,2-distearoyl-sn-glycero-3-phosphocholine, cholesterol, DL-dipalmitoylphosphatidylglycerol, sodium N-(carbonyl-methoxypolyethylene glycol 2000)-1,2-distearoyl-sn-glycero-3-phosphoethanolamine, sodium N-(carbonyl-methoxypolyethylene glycol 2000)-distearoyl-glycerophosphoethanolamine, DL-distearoylphosphatidylcholine, egg phospholipids, and hydrogenated soybean lecithin.

In some embodiments, the at least one encapsulation agent is 1,2-dimyristoyl-sn-glycero-phosphocholine. In some embodiments, the at least one encapsulation agent is 1,2-distearoyl-sn-glycero-3-(phosphor-rac-(1-glycerol)). In some embodiments, the at least one encapsulation agent is 1,2-distearoyl-sn-glycero-3-phosphocholine. In some embodiments, the at least one encapsulation agent is cholesterol. In some embodiments, the at least one encapsulation agent is DL-dipalmitoylphosphatidylglyce-rol. In some embodiments, the at least one encapsulation agent is sodium N-(carbonyl-methoxypolyethylene glycol 2000)-1,2-distearoyl-sn-glycero-3-phosphoethanolamine. In some embodiments, the at least one encapsulation agent is sodium N-(carbonyl-methoxypolyethylene glycol 2000)-distearoyl-glycerophosphoethanolamine. In some embodiments, the at least one encapsulation agent is DL-distearoylphosphatidylcholine. In some embodiments, the at least one encapsulation agent is an egg phospholipid. In some embodiments, the at least one encapsulation agent is hydrogenated soybean lecithin.

In some embodiments, the at least one encapsulation agent is present in an amount ranging from 0.005% to 10% by weight of the composition. In some embodiments, the at least one encapsulation agent is present in an amount ranging from 0.005% to 0.1%, 0.25%, 0.5%, 1%, 1.5%, 2.5%, 5%, 7.5%, or 10% by weight of the composition. In some embodiments, the at least one encapsulation agent is present in an amount ranging from 0.1% to 10% by weight of the composition. In some embodiments, the at least one encapsulation agent is present in an amount ranging from 0.1% to 0.25%, 0.5%, 1%, 1.5%, 2.5%, 5%, 7.5%, or 10% by weight of the composition.

In some embodiments, the at least one encapsulation agent is present in an amount ranging from 1% to 10% by weight of the composition. In some embodiments, the at least one encapsulation agent is present in an amount ranging from 1% to 1.5%, 2.5%, 5%, 7.5%, or 10% by weight of the composition.

In some embodiments, the at least one encapsulation agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one encapsulation agent is present in an amount of 0.005% by weight of the composition. In some embodiments, the at least one encapsulation agent is present in an amount of 0.01% by weight of the composition. In some embodiments, the at least one encapsulation agent is present in an amount of 0.025% by weight of the composition. In some embodiments, the at least one encapsulation agent is present in an amount of 0.05% by weight of the composition. In some embodiments, the at least one encapsulation agent is present in an amount of 0.1% by weight of the composition. In some embodiments, the at least one encapsulation agent is present in an amount of 0.15% by weight of the composition. In some embodiments, the at least one encapsulation agent is present in an amount of 0.2% by weight of the composition. In some embodiments, the at least one encapsulation agent is present in an amount of 0.25% by weight of the composition. In some embodiments, the at least one encapsulation agent is present in an amount of 0.3% by weight of the composition. In some embodiments, the at least one encapsulation agent is present in an amount of 0.4% by weight of the composition. In some embodiments, the at least one encapsulation agent is present in an amount of 0.5% by weight of the composition. In some embodiments, the at least one encapsulation agent is present in an amount of 0.75% by weight of the composition. In some embodiments, the at least one encapsulation agent is present in an amount of 1% by weight of the composition. In some embodiments, the at least one encapsulation agent is present in an amount of 1.5% by weight of the composition. In some embodiments, the at least one encapsulation agent is present in an amount of 2% by weight of the composition. In some embodiments, the at least one encapsulation agent is present in an amount of 2.5% by weight of the composition. In some embodiments, the at least one encapsulation agent is present in an amount of 5% by weight of the composition. In some embodiments, the at least one encapsulation agent is present in an amount of 7.5% by weight of the composition. In some embodiments, the at least one encapsulation agent is present in an amount of 10% by weight of the composition.

### At Least One Imaging Agent

In some embodiments, the composition further comprises at least one imaging agent. In some embodiments, the at least one imaging agent is a radiographic contrast agent. In some embodiments, when at least one imaging agent is present in the composition, the composition further comprising at least one imaging agent is formed by combining a composition comprising at least one imaging agent and a composition comprising at least one active agent. In some embodiments, the composition comprising at least one active agent is a composition described herein.

In some embodiments, when at least one imaging agent is present in the composition, the composition further comprising at least one imaging agent is formed by combining a composition comprising at least one imaging agent and a composition comprising at least one active agent prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 24 hours prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 6 hours prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent 15 seconds to 1 hour prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent less than 2 hours prior to use of the composition comprising at least one active agent and further comprising at least one imaging agent.

In some embodiments, when at least one imaging agent is present in the composition, the ratio of the volume of a composition comprising at least one imaging agent to the volume of a composition comprising at least one active agent ranges from 0.5:1 to 2.5:1 prior to combination. In some embodiments, when at least one imaging agent is present in the composition, the ratio of the volume of a composition comprising at least one imaging agent to the volume of a composition comprising at least one active agent ranges from 0.5:1 to 1.5:1 prior to combination. In some embodiments, when at least one imaging agent is present in the composition, the ratio of the volume of a composition comprising at least one imaging agent to the volume of a composition comprising at least one active agent ranges from 0.75:1 to 1.25:1 prior to combination.

In some embodiments, when at least one imaging agent is present in the composition, the ratio of the volume of a composition comprising at least one imaging agent to the volume of a composition comprising at least one active agent is 0.5:1 prior to combination. In some embodiments, when at least one imaging agent is present in the composition, the ratio of the volume of a composition comprising at least one imaging agent to the volume of a composition comprising at least one active agent is 0.75:1 prior to combination. In some embodiments, when at least one imaging agent is present in the composition, the ratio of the volume of a composition comprising at least one imaging agent to the volume of a composition comprising at least one active agent is 1:1 prior to combination. In some embodiments, when at least one imaging agent is present in the composition, the ratio of the volume of a composition comprising at least one imaging agent to the volume of a composition comprising at least one active agent is 1.25:1 prior to combination. In some embodiments, when at least one imaging agent is present in the composition, the ratio of the volume of a composition comprising at least one imaging agent to the volume of a composition comprising at least one active agent is 1.5:1 prior to combination. In some embodiments, when at least one imaging agent is present in the composition, the ratio of the volume of a composition comprising at least one imaging agent to the volume of a composition comprising at least one active agent is 1.75:1 prior to combination. In some embodiments, when at least one imaging agent is present in the composition, the ratio of the volume of a composition comprising at least one imaging agent to the volume of a composition comprising at least one active agent is 2:1 prior to combination. In some embodiments, when at least one imaging agent is present in the composition, the ratio of the volume of a composition comprising at least one imaging agent to the volume of a composition comprising at least one active agent is 2.25:1 prior to combination. In some embodiments, when at least one imaging agent is present in the composition, the ratio of the volume of a composition comprising at least one imaging agent to the volume of a composition comprising at least one active agent is 2.5:1 prior to combination.

In some embodiments, when at least one imaging agent is present in the composition, the molar ratio of the at least one imaging agent to the at least one active agent ranges from 300:1 to 50:1. In some embodiments, when at least one imaging agent is present in the composition, the molar ratio of the at least one imaging agent to the at least one active agent ranges from 250:1 to 75:1. In some embodiments, when at least one imaging agent is present in the composition, the molar ratio of the at least one imaging agent to the at least one active agent ranges from 200:1 to 100:1. In some embodiments, when at least one imaging agent is present in the composition, the molar ratio of the at least one imaging agent to the at least one active agent ranges from 175:1 to 125:1. In some embodiments, when at least one imaging agent is present in the composition, the molar ratio of the at least one imaging agent to the at least one active agent is 158:1.

### At Least One Anti-Proliferative Agent

In some embodiments, the composition further comprises at least one anti-proliferative agent. In some embodiments, when at least one anti-proliferative agent is present in the composition, the composition further comprising at least one anti-proliferative agent is formed by combining a composition comprising at least one anti-proliferative agent and a composition comprising at least one active agent. In some embodiments, the composition comprising at least one active agent is a composition described herein.

In some embodiments, when at least one anti-proliferative agent is present in the composition, the composition further comprising at least one anti-proliferative agent is formed by combining a composition comprising at least one anti-proliferative agent and a composition comprising at least one active agent prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 24 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 6 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 1 hour prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent less than 2 hours prior to use.

In some embodiments, the at least one anti-proliferative agent is chosen from paclitaxel, paclitaxel derivatives, rapamycin, rapamycin derivatives, and pharmaceutically acceptable salts thereof. In some embodiments, the at least one anti-proliferative agent is paclitaxel. In some embodiments, the paclitaxel derivatives are chosen from docetaxel, and cabazitaxel. In some embodiments, the at least one anti-proliferative agent is rapamycin. In some embodiments, the rapamycin derivatives are chosen from everolimus, ridaforolimus, tacrolimus, umirolimus, and zotarolimus.

In some embodiments, when at least one anti-proliferative agent is present in the composition, the ratio of the volume of a composition comprising at least one anti-proliferative agent to the volume of a composition comprising at least one active agent ranges from 0.5:1 to 2.5:1 prior to combination. In some embodiments, when at least one anti-proliferative agent is present in the composition, the ratio of the volume of a composition comprising at least one anti-proliferative agent to the volume of a composition comprising at least one active agent ranges from 0.5:1 to 1.5:1 prior to combination. In some embodiments, when at least one anti-proliferative agent is present in the composition, the ratio of the volume of a composition comprising at least one anti-proliferative agent to the volume of a composition comprising at least one active agent ranges from 0.75:1 to 1.25:1 prior to combination.

In some embodiments, when at least one anti-proliferative agent is present in the composition, the ratio of the volume of a composition comprising at least one anti-proliferative agent to the volume of a composition comprising at least one active agent is 0.5:1 prior to combination. In some embodiments, when at least one anti-proliferative agent is present in the composition, the ratio of the volume of a composition comprising at least one anti-proliferative agent to the volume of a composition comprising at least one active agent is 0.75:1 prior to combination. In some embodiments, when at least one anti-proliferative agent is present in the composition, the ratio of the volume of a composition comprising at least one anti-proliferative agent to the volume of a composition comprising at least one active agent is 1:1 prior to combination. In some embodiments, when at least one anti-proliferative agent is present in the composition, the ratio of the volume of a composition comprising at least one anti-proliferative agent to the volume of a composition comprising at least one active agent is 1.25:1 prior to combination. In some embodiments, when at least one anti-proliferative agent is present in the composition, the ratio of the volume of a composition comprising at least one anti-proliferative agent to the volume of a composition comprising at least one active agent is 1.5:1 prior to combination. In some embodiments, when at least one anti-proliferative agent is present in the composition, the ratio of the volume of a composition comprising at least one anti-proliferative agent to the volume of a composition comprising at least one active agent is 1.75:1 prior to combination. In some embodiments, when at least one anti-proliferative agent is present in the composition, the ratio of the volume of a composition comprising at least one anti-proliferative agent to the volume of a composition comprising at least one active agent is 2:1 prior to combination. In some embodiments, when at least one anti-proliferative agent is present in the composition, the ratio of the volume of a composition comprising at least one anti-proliferative agent to the volume of a composition comprising at least one active agent is 2.25:1 prior to combination. In some embodiments, when at least one anti-proliferative agent is present in the composition, the ratio of the volume of a composition comprising at least one anti-proliferative agent to the volume of a composition comprising at least one active agent is 2.5:1 prior to combination.

In some embodiments, when at least one anti-proliferative agent is present in the composition, the molar ratio of the at least one anti-proliferative agent to the at least one active agent ranges from 0.25:4 to 4:0.25. In some embodiments, when at least one anti-proliferative agent is present in the composition, the molar ratio of the at least one anti-proliferative agent to the at least one active agent ranges from 0.75:2 to 2:0.75. In some embodiments, when at least one anti-proliferative agent is present in the composition, the molar ratio of the at least one anti-proliferative agent to the at least one active agent ranges from 0.75:1.5 to 1.25:1.5. In some embodiments, when at least one anti-proliferative agent is present in the composition, the molar ratio of the at least one anti-proliferative agent to the at least one active agent ranges from 0.9:1.3 to 1.1:1.5. In some embodiments, when at least one anti-proliferative agent is present in the composition, the molar ratio of the at least one anti-proliferative agent to the at least one active agent is 1:1.4.

### Composition Properties

### Composition pH

In some embodiments, the composition has a pH ranging from 4 to 8. In some embodiments, the composition has a pH ranging from 5 to 7. In some embodiments, the composition has a pH ranging from 5.5 to 6.5.

Without being bound by any theory, in some embodiments, a composition having a pH ranging from 5 to 7 may be suitable for intraarterial or intravenous delivery and/or may result in a composition where the at least one active agent does not degrade after a period of time compared to a composition having a pH outside of that range, as described herein below.

In some embodiments, the composition has a pH of 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7. In some embodiments, the composition has a pH of 5. In some embodiments, the composition has a pH of 5.1. In some embodiments, the composition has a pH of 5.2. In some embodiments, the composition has a pH of 5.3. In some embodiments, the composition has a pH of 5.4. In some embodiments, the composition has a pH of 5.5. In some embodiments, the composition has a pH of 5.6. In some embodiments, the composition has a pH of 5.7. In some embodiments, the composition has a pH of 5.8. In some embodiments, the composition has a pH of 5.9. In some embodiments, the composition has a pH of 6. In some embodiments, the composition has a pH of 6.1. In some embodiments, the composition has a pH of 6.2. In some embodiments, the composition has a pH of 6.3. In some embodiments, the composition has a pH of 6.4. In some embodiments, the composition has a pH of 6.5. In some embodiments, the composition has a pH of 6.6. In some embodiments, the composition has a pH of 6.7. In some embodiments, the composition has a pH of 6.8. In some embodiments, the composition has a pH of 6.9. In some embodiments, the composition has a pH of 7.

### Non-Limiting Exemplary Composition Embodiments

In some embodiments, provided herein is a composition comprising at least one active agent; at least one tonicity agent; at least one buffer; and at least one vehicle.

In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6, and wherein the at least one active agent does not degrade 156 weeks after preparation of the composition.

In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water. In some embodiments, the composition has a pH of 6. In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition.

In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one imaging agent comprises a radiographic contrast agent. In some embodiments, the composition further comprises at least one anti-proliferative agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one active agent does not degrade 24 hours after preparation of the composition. In some embodiments, degradation is determined by LC. In some embodiments, the composition further comprises at least one active agent. In some embodiments, the composition further comprises at least one anti-proliferative agent.

In some embodiments, the at least one active agent is chosen from a Compound of Formula (I) and pharmaceutically acceptable salts thereof. In some embodiments, the at least one active agent is a diacetate salt of a Compound of Formula (I).

In some embodiments, the at least one active agent is a diacetate salt of a Compound of Formula (I) present in an amount of 0.01% to 1% by weight of the composition. In some embodiments, the at least one active agent is a diacetate salt of a Compound of Formula (I) present in an amount of 0.01% to 1% by weight of the composition and the at least one tonicity agent is chosen from potassium chloride and sodium chloride. In some embodiments, the at least one active agent is a diacetate salt of a Compound of Formula (I) present in an amount of 0.01% to 1% by weight of the composition and the at least one tonicity agent is chosen from potassium chloride and sodium chloride present in an amount ranging from 0.5% to 1.5% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic present in an amount of 0.1% to 0.16% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, wherein the composition has a pH of 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, wherein the pH of the composition is 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition, wherein the composition has a pH of 6.

### Visible Light

The methods disclosed herein comprise illuminating the at least one active agent on the AVF with visible light, such as, for example, 450 nm light. The source of visible light may be, for example, chosen from lamps, light emitting diodes, and other sources of electromagnetic radiation. Light can also be supplied to an internal surface or tissue using an optical fiber device, e.g., the light can be delivered by optical fibers threaded through a small gauge hypodermic needle or an arthroscope. Light can also be transmitted by percutaneous instrumentation using optical fibers or cannulated waveguides.

### Biological Processes

In some embodiments, treatment with the compositions disclosed herein comprise preserving ECM integrity and/or preventing inflammation at the AVF. In some embodiments, the treatment methods disclosed herein do not change the expression level of MMP-2 in the treated AVF. In some embodiments, the treatment methods disclosed herein decrease the expression level of MMP-2 in the treated AVF in comparison to the expression level of MMP-2 in an untreated AVF. In some embodiments, the treatment methods disclosed herein do not change the expression level of MMP-9 in the treated AVF. In some embodiments, the treatment methods disclosed herein decrease the expression level of MMP-9 in the treated AVF in comparison to the expression level of MMP-9 in an untreated AVF. In some embodiments, the treatment methods disclosed herein do not change the expression level of IL-6 in the treated AVF. In some embodiments, the treatment methods disclosed herein decrease the expression level of IL-6 in the treated AVF in comparison to the expression level of IL-6 in an untreated AVF.

In some embodiments, treatment with the compositions disclosed herein does not cause or increase cell death in the treated AVF in comparison to an untreated AVF. In some embodiments, treatment with the compositions disclosed herein does not cause or increase vascular wall cell proliferation in the treated AVF in comparison to an untreated AVF. In some embodiments, treatment with the compositions disclosed herein does not increase the expression level of Ki-67 in the treated AVF in comparison to an untreated AVF.

In some embodiments, treatment with the compositions disclosed herein causes outward remodeling of the vessels of the treated AVF. In some embodiments, treatment with the compositions disclosed herein significantly increases the AVF open lumen area. In some embodiments, treatment with the compositions disclosed herein significantly increases the AVF open lumen area without significantly affecting the neointimal hyperplasia area. In some embodiments, treatment with the compositions disclosed herein significantly increase the AVF venous diameter. In some embodiments, treatment with the compositions disclosed herein significantly increases the AVF venous diameter after the AVF creation surgery. In some embodiments, treatment with the compositions disclosed herein significantly increases the AVF venous diameter by 4 weeks after the AVF creation surgery.

In order that the disclosure herein may be more fully understood, the following examples are set forth. It should be understood that these examples are for illustrative purposes only and are not to be construed as limiting this disclosure in any manner.

### EXAMPLES

### Example 1

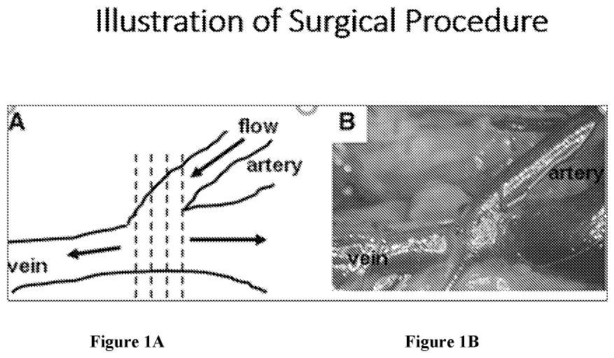

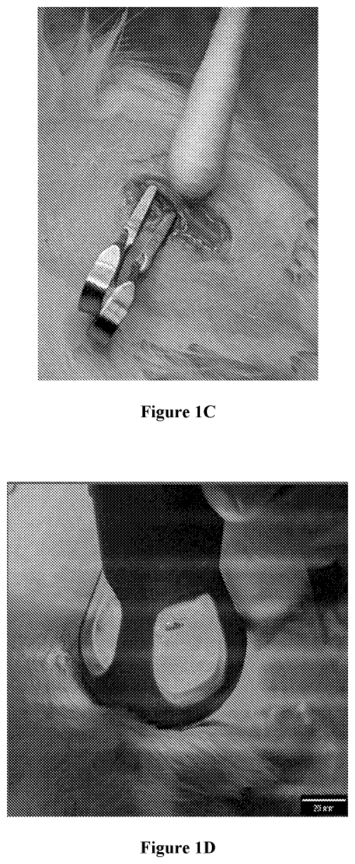

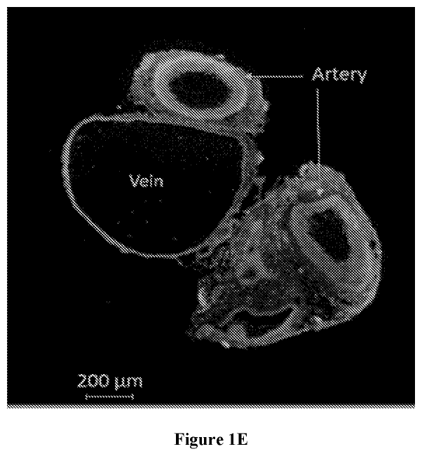

Femoral AVFs were created in young Wistar male rats by connecting the femoral artery (side) and femoral vein (end) in the same limb. FIGS. 1A-B show the surgical procedure. Immediately after the blood flow was restored and dilatation of the femoral vein occurred, a 10 μl -drop of a composition comprising 2 mg/ml Compound of Formula (I) in PBS was placed at the AVF perivascularly and incubated for 5 minutes, to allow the Compound of Formula (I) to diffuse into the vascular wall. The Compound of Formula (I) was activated by a 1 minute illumination by 450 nm light. A control group of young Wistar male rats received a 10 μl -drop of phosphate buffered saline and the same light activation. The rats' skin was closed immediately after light activation. FIG. 1C is a photograph of the incubation with NVS, applied perivascularly on the anastomosis to allow the compound to diffuse into the vascular tissue. FIG. 1D is a photograph of the small molecule treated area being exposed to 1 minute of illumination with a 450 -nm light source. FIG. 1E depicts fluorescence images after following light activation, 2 animals were sacrificed and their vessels observed on a fluorescence microscope at 450 nm excitation for NVS.

Rats were euthanized 1 week (early time point, 4 per group) and 4 weeks (later time point, 10 per group) post-AVF creation for histology and morphometry. Verhoeff-Van Gieson (VVG)-stained thin cross-sections were used to measure the area enclosed by internal elastic lamina (IEL), the area of open lumen, and the area of neointimal hyperplasia (NH) by Image J software. Percent open lumen area=open lumen area/IEL-enclosed area×100%. These analyses were done at three locations: the entire AVF, the AVF venous limb, and the AVF arterial limb.

Results: The animals in both experimental groups tolerated the treatment well.

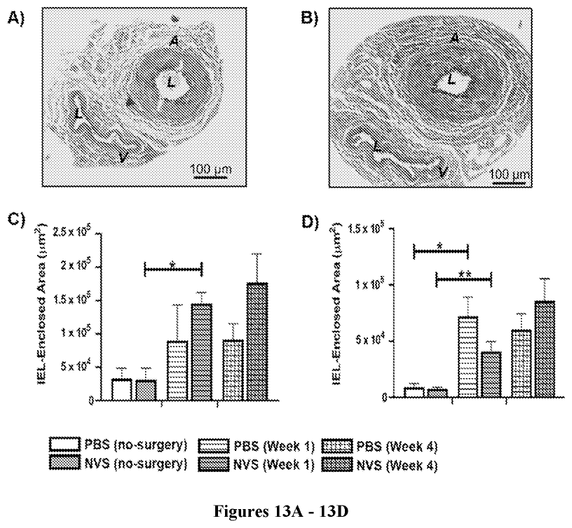

The pre-surgery IEL-enclose area in the vein was similar between the PBS and NVS groups (FIG. 13C, 32865 vs. 31499 μm², p=0.954). The pre-surgery IEL-enclose area in the artery was also similar between the PBS and NVS groups (FIG. 13D, 9060 vs. 7613 μm², p=0.703). At 1 week after AVF creation, both vein and artery areas significantly increased when compared to their pre-surgical values (FIG. 13), suggesting successful AVG creation surgery in both PBS and NVS groups.

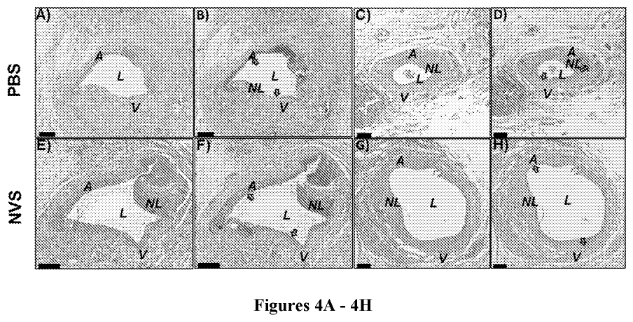

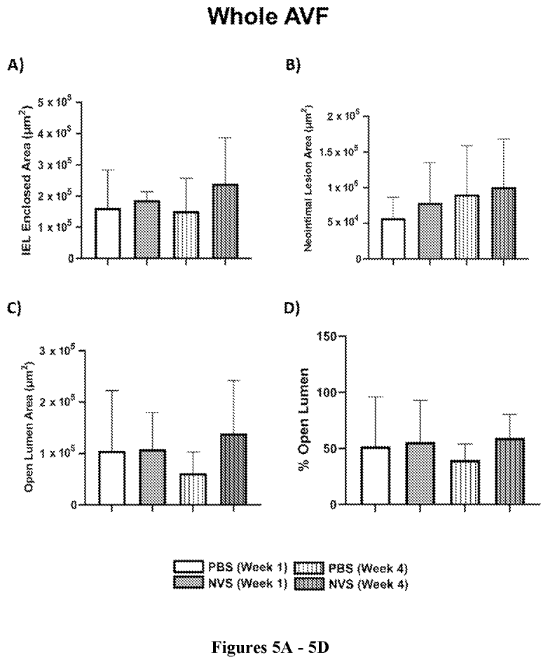

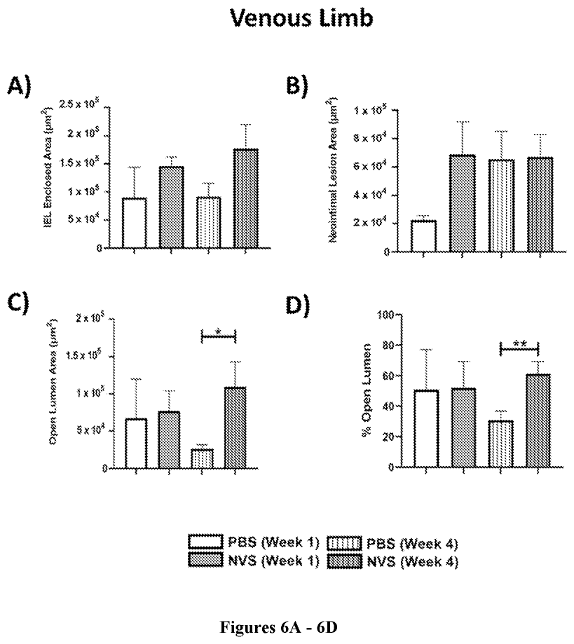

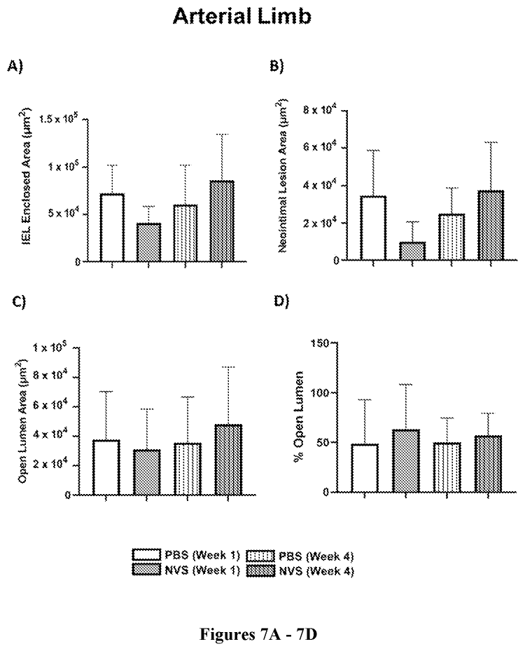

FIG. 4 displays representative histology images of the entire AVF. The IEL-enclosed area represents the maximal possible open lumen area without any neointimal lesion, because in a normal vessel the intimal layer is very thin, with just a single layer of endothelial cells. At 1 week, the entire AVF's lumen looked similar between treated and control groups. All four morphometric parameters (the IEL-enclosed area, the neointimal lesion area, the open lumen area, and the % area of open lumen) in the entire AVF were similar between NVS-treated and PBS-treated AVFs after 1 week. At 4 weeks, however, the entire AVF's open lumen area and % open lumen area were trending larger in treated rats than in control rats, being 2.25 and 1.49-fold larger, respectively (p=0.062, p=0.052) (FIG. 5). When the venous and arterial sides of the AVF were considered, the difference in open lumen area occurred in the venous side, where the AVF venous limb's open lumen area and % open lumen area in treated rats were 4.18 and 1.98-fold larger than in control rats, respectively (p=0.014, p=0.009) (FIG. 6). The AVF arterial limb's open lumen area was similar in both groups (FIG. 7). The NH area was similar in both groups, regardless of the locations. These differences indicate that the NVS compound potentially improves AVF patency by improving outward remodeling in the vein.

The rat 1-week and 4-week AVF morphometric analysis data for each of the total AVF (only 4-week data) (FIG. 5), AVF vein (FIG. 6), and AVF artery (FIG. 7) including:

IEL-enclosed area

Open lumen area

NH area, and

percentage of open lumen

were plotted as shown in FIGS. 5-7 as column graphs. An unpaired, two-tailed t-test was used to determine the statistical significance between the two treatment groups (no-NVS (i.e., control) vs. NVS (treatment with Compound of Formula (I))) in each of the data sets, with α=0.05.

The “AVF total open lumen area,” “AVF total fraction of open lumen,” “AVF vein open lumen area,” and “AVD vein percentage of open lumen” of the no-NVS and NVS group were significantly different from each other.

In summary, rats tolerated treatment well and the treatment significantly increased the AVF open lumen area, without significantly affecting the NH area.

These studies show that when compared to the PBS control groups, NVS therapy did not affect the neointimal lesion area (i.e., no effect on inward remodeling) but led to an enlarged venous diameter (i.e., better outward remodeling) by 4 weeks after the AVF creation surgery.

### Example 2

Parameters analyzed for morphometry included:

AVF total IEL area

AVF total % occluded lumen (calculated from equation: [AVF total IEL area−AVF total open lumen area]/AVF total IEL area)

AVF artery IEL area

AVF artery % occluded lumen (calculated from equation: [AVF artery IEL area−AVF artery open lumen area]/AVF artery IEL area)

AVF vein IEL area

AVF vein % occluded lumen (calculated from equation: [AVF vein IEL area−AVF vein open lumen area]/AVF vein IEL area)

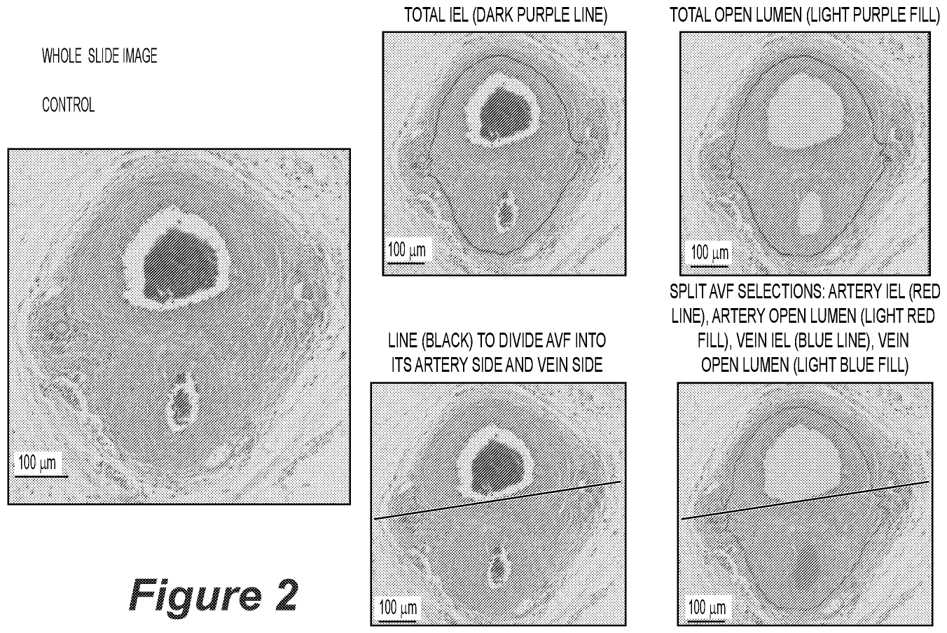

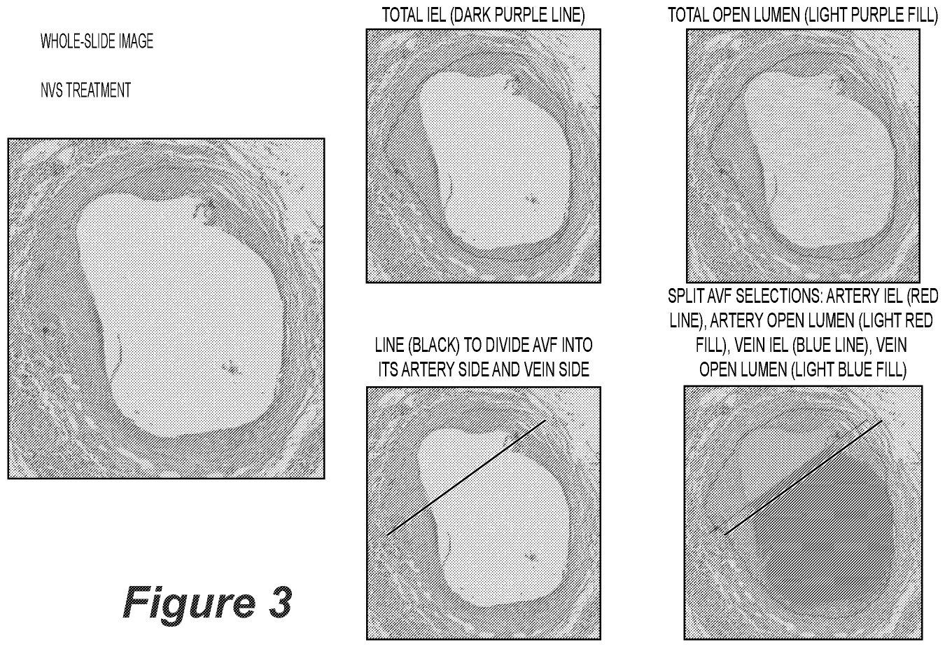

FIGS. 2 and 3 show VVG images of a control rat and a rat treated with Compound of Formula (I) with regions of interest (ROIs) drawn in to measure the above underlined parameters. Many IEL parts (in both the arterial and venous side of the AVFs) were missing, most likely due to vascular remodeling. Those missing parts had to be estimated for drawing the ROIs.

### Example 3: Immunohistochemistry of the AVF

Based on the results from the morphometric analysis in Example 1, the expression level of several molecular targets associated with AVF maturation failure (i.e., MMP2-MMP-9, Ki-67, and IL6) in the AVF vessels at the 4-week time point were selected for further investigation.

During the AVF maturation process, there is significantly increased expression of extracellular matrix (ECM) components, regulatory proteins such as MMP and TIMP and structural proteins such as collagen and elastin, mediating a controlled pattern of ECM remodeling without structural failure. But excessive fibrosis of the vessel walls due to local inflammation as well as the circumferential arrangement of newly synthesized collagen fibers has shown a positive association with AVF non-maturation (possibly due to decreasing the distensibility of the veins as they go through the arterialization process). MMP-9 is an inflammatory marker, and although MM-2 is usually not considered an inflammatory marker, both metalloproteinases have nearly identical ECM substrates and are important in vascular remodeling. Some studies have suggested a negative association between MMP-2/MMP-9 and AVF maturation. Ki-67 is a marker of cell proliferation, which allows assessing the proliferation of vascular wall cells during the AVF development process. IL-6 is an inflammatory cytokine, whose receptor activation has been suggested to have a role in the pathogenesis of AVF failure in hemodialysis patients.

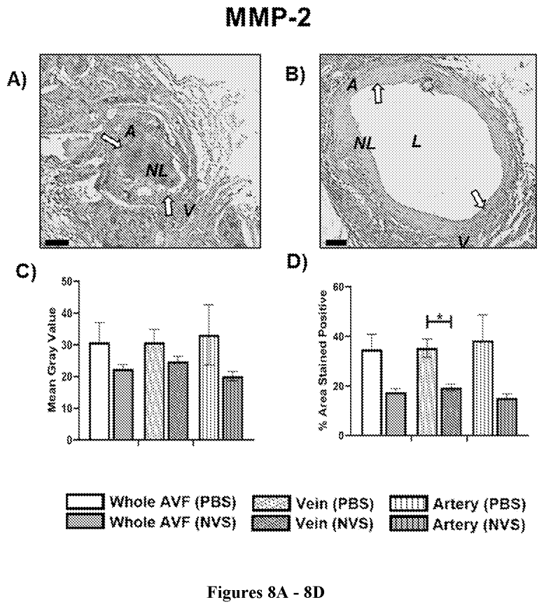

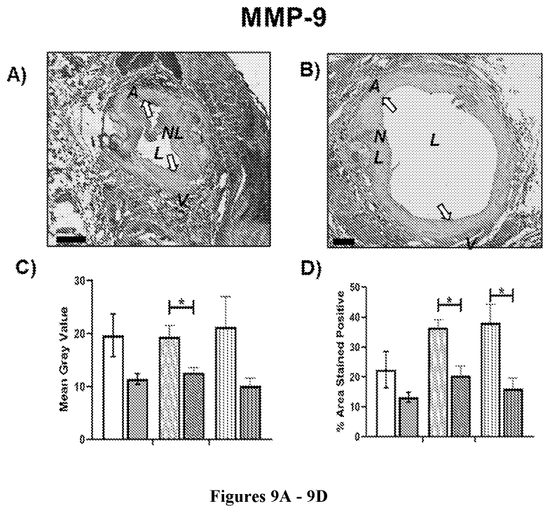

FIGS. 8 and 9 display representative IHC images and quantitative results of MMP-2 and MMP-9 analysis. The MMP-2 expression level was trending lower in the NVS group than in the PBS group in the entire AVF and the arterial limb; and, MMP-2 in the venous limb was significantly lower in the NVS group than the PBS group in terms of area stained positive (FIG. 8C, 1.8-fold, p=0.015). In the entire AVF, the MMP-9 expression level was trending lower in the NVS group than in the PBS group; and, MMP-9 in the venous limb (FIG. 9D, 1.8-fold, p=0.019) and arterial limb (FIG. 9D, 2.4-fold, p=0.036) was significantly lower in the NVS group than the PBS group in terms of % area stained positive. Thus, NVS treatment caused an increased in vessel lumen size that was accompanied by decreased MMP-2 and MMP-9 expression levels, suggesting that NVS prevents inflammation and ECM remodeling associated with AVF failure.

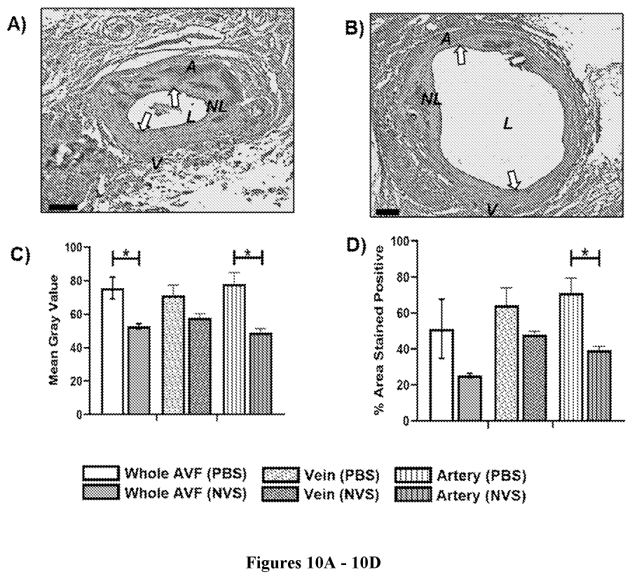

FIG. 10 displays representative IHC images and quantitative result of IL-6 analysis. The IL-6 expression level was trending lower in the NVS group than in the PBS group in the venous limb. Additionally, IL-6 in the entire AVF (FIG. 10C, 1.4-fold, p=0.027) and arterial limb (FIG. 10C, 1.6-fold, p=0.015) was significantly lower in the NVS group than the PBS in terms of mean gray value. Similar to the finding of a decreased MMP-9 expression level in NVS-treated groups, a decreased IL-6 expression level in the NVS-treated groups suggests that NVS prevents inflammation mechanisms associated with AVF failure.

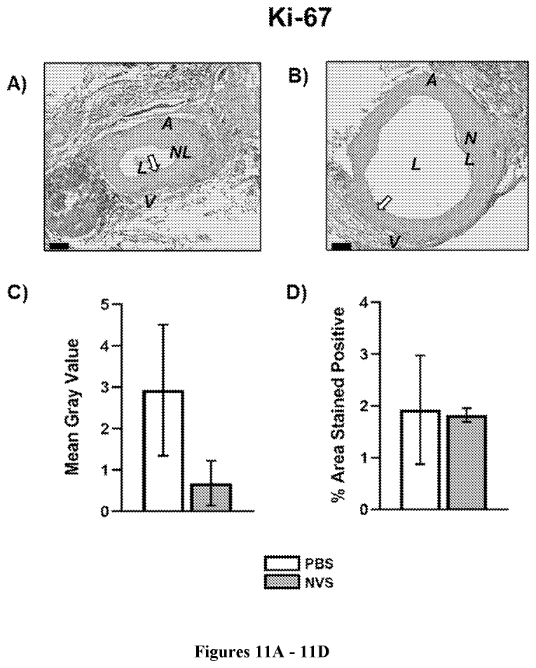

FIG. 11 displays representative IHC images and quantitative results of Ki-67 analysis. The Ki-67 expression level was similar in the NVS group and the PBS group in the entire AVF. This is consistent with the notion that NVS therapy does not affect the proliferation of vascular wall cells during the AVF development process. In this context this proliferation is referred to as “arterialization.” An aspect of the present invention is that it does not impair arterialization of the vein in the AVF, which is a key process in fistula maturation.

### Example 4: Cell Death Analysis

Cell death assays used the cell death detection R&D system kit to detect cell death via apoptosis. Cell density was 1×10⁵ per well in a 96-well plate for R&D system kit. HUVECs and HASMCs were treated with NVS in different concentrations (0, 0.25, 2.5, and 25 μg/ml) in serum free medium for 5 minutes in the incubator. After removing NVS, cells were washed with serum free medium once, and the plate was refilled with 200 μl serum free medium in each well. Cells were treated using 450 nm light for 1 minute at room temperature, and then serum free medium was removed and each well was refilled with 200 μl of medium supplemented with 10% serum. At 0, 1, 2, and 3 days post-treatment (D0, D1, D2, and D3), cell death assay was performed according to the manufacturer's protocol. Positive controls were cells incubated with hypertonic buffer (10 mM Tris PH 7.4, 400 mM NaCl, 10 mM MgCl2) at 37° C. for 2 hours.

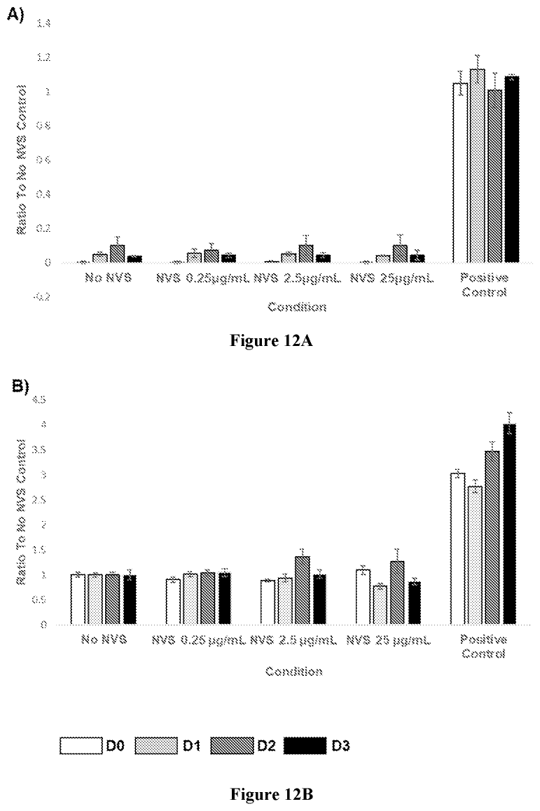

NVS treatment caused minimal cell death via apoptosis using the R&D systems kits (FIG. 12). This suggests that NVS does not have detrimental cytotoxic effects on blood vessel cells such as endothelial or smooth muscle cells, further showing it is safe for treatment.

### Example 5: Multiphoton imaging of Collagen Fibers

Second harmonic generation (SHG) imaging was performed on 5 μm thick formalin-fixed paraffin embedded (FFPE) sections using Leica SP8 Dive with spectral tunable detection. Collagen fiber orientation in the medial layer was analyzed in relationship to the lumen. Each sample was divided into 4 quadrants and the medial layer was examined in each quadrant; the area of examination for each quadrant was 25 μm². The same images were used for other collagen morphology using the MIPAR image software analysis.

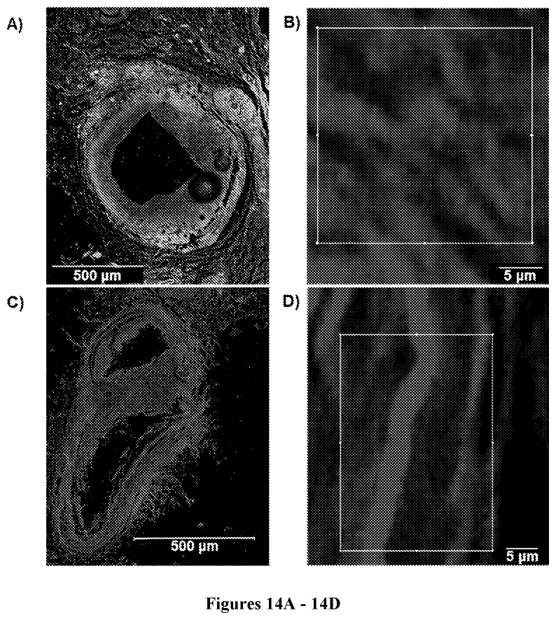

Collagen direction is considered favorable for AVF maturation if it is perpendicular to the lumen. SHG images of the AVFs at week 4 were acquired (FIG. 14). 58% of analyzed regions in the NVS treated samples exhibited favorable collagen orientation for AVF maturation, whereas only 25% of analyzed regions in the PBS treated samples exhibited a favorable collagen orientation, though this difference is not statistically significant. Collagen morphological data showed a trend for NVS treated samples to have less overall collagen area yet more defined shape features (i.e., roundness, roughness, and eccentricity) than PBS treated samples, although not statistically different.

What is claimed:

1. A method for maturing an arteriovenous fistula (AVF) comprising:
administering to an AVF a composition comprising at least one active agent chosen from a Compound of Formula (I): 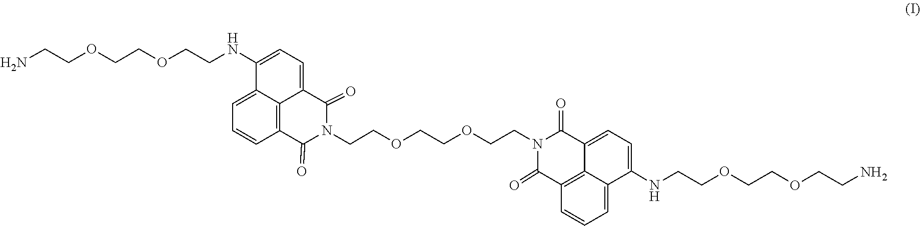 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof; and
illuminating the at least one active agent on said AVF with visible light.

2. The method according to claim 1, wherein the AVF open lumen area is increased without statistically significantly affecting the neointimal hyperplasia area.

3. The method according to claim 1, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium).

4. The method according to claim 1, wherein the at least one active agent is present in an amount ranging from 0.01% to 4% by weight of the composition.

5. The method according to claim 1, wherein the composition further comprises at least one solvent.

6. The method according to claim 1, wherein the at least one active agent on said AVF is illuminated with visible light after incubation for 3-10 minutes.

7. The method according to claim 1, wherein the at least one active agent on said AVF is illuminated with light at 450 nm for a period sufficient to activate said at least one active agent.

8. The method according to claim 1 or 7, wherein the period sufficient to activate said at least one active agent is 30 seconds to 5 minutes.

9. The method according to claim 1 or 7, wherein the period sufficient to activate said at least one active agent is 1 minute to 2 minutes.

10. The method according to claim 1, wherein the composition is administered to the AVF at the time of fistula creation.

11. The method of any one of claims 1-10, wherein the AVF venous diameter is enlarged.

12. The method of claim 11, wherein the AVF venous diameter is enlarged due to an outward remodeling mechanism.

13. The method of any one of claims 1-12, wherein the AVF venous diameter is enlarged by 4 weeks after AVF creation surgery.

14. The method of any one of claims 1-13, wherein the proliferation of vascular wall cells is not affected during AVF maturation.

15. The method of any one of claims 1-14, wherein the expression level of Ki-67 in the AVF is lower than or similar to the expression level of Ki-67 in an AVF not administered the composition.

16. The method of any one of claims 1-15, wherein the expression level of MMP-9 in the AVF is lower than the expression level of MMP-9 in an AVF not administered the composition.

17. The method of any one of claims 1-16, wherein the expression level of MMP-2 in the AVF is lower than the expression level of MMP-2 in an AVF not administered the composition.

18. The method of any one of claims 1-17, wherein the expression level of IL-6 in the AVF is lower than the expression level of IL-6 in an AVF not administered the composition.

19. The method of any one of claims 1-18, wherein no substantial cell death occurs in the AVF after treatment.

20. The method of any one of claims 1-19, wherein the collagen orientation in the AVF is favorable for AVF maturation.
